(19) United States
(12) Patent Application Publication
(10) Pub. No.: US 2011/0142882 A1
(43) Pub. Date: Jun. 16, 2011

(54) Control of blood flow in skin, subcutaneous tissue and muscle by botulinum toxin, snare protein mediating substances, and alpha adrenergic agents by either injection of transdermal delivery systems

(75) Inventors: L. Andrew Koman, Winston-Salem, NC (US); Thomas Smith, Winston-Salem, NC (US)

(73) Assignee: DT SCIMED, LLC

(21) Appl. No.: 12/654,281

(22) Filed: Dec. 16, 2009

(51) Int. Cl. A61K 39/08; A61P 17/00

(52) U.S. Cl. 424/239.1

(57) ABSTRACT

Methods for controlling abnormal blood flow in disease states by the selective alteration of the components of microvascular flow using Botulinum A Toxin, other SNARE protein mediating agents, alpha adrenergic agents, and other compounds is heretofore unknown, whether delivered by injection, topical application, or transdermal means. The invention discloses novel methods for the control of blood flow in skin, subcutaneous tissue and muscle by botulinum toxin, snare protein mediating substances, and alpha adrenergic agents by either injection or transdermal delivery systems.

### RELATED APPLICATIONS

There are no related applications.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

None.

### FIELD OF THE INVENTION

This invention relates to a dosing protocol for the administration of botulinum toxin for the control of blood flow in skin, subcutaneous tissues, and muscle.

### BACKGROUND OF THE INVENTION

The use of botulinum toxins, in particular botulinum toxin type A, in the treatment of neuromuscular disorders and conditions involving muscular spasm as well as in cosmetic procedures is known. Known methods include treatment regimes for strabismus, blepharospasm, spasmodic torticollis (cervical dystonia), oromandibular dystonia and spasmodic dysphonia (laryngeal dystonia), as well as the now common cosmetic procedures for reducing wrinkles, and to a lesser extent, reducing sweating. The toxin binds rapidly and strongly to presynaptic nerve terminals and inhibits the exocytosis of neurotransmitters by decreasing the frequency of transmitter release thereby reducing or eliminating the activation of postsynaptic muscles, nerves, or effector tissues. This results in local paralysis and hence relaxation of the afflicted muscle.

The term botulinum toxin as used herein is a generic term embracing the family of toxins produced by the anaerobic bacterium Clostridium botulinum and, to date, seven immunologically distinct toxins have been identified. These have been given the designations A, B, C, D, E, F and G. For further information concerning the properties of the various botulinum toxins, reference is made to the article by Jankovic & Brin, The New England Journal of Medicine, pp 1186-1194, No 17, 1991 and to the review by Charles L Hatheway, Chapter 1 of the book entitled Botulinum Neurotoxin and Tetanus Toxin Ed. L. L. Simpson, published by Academic Press Inc. of San Diego Calif. 1989, the disclosures of which are incorporated herein by reference.

The neurotoxic component of botulinum toxin has a molecular weight of about 150 kilodaltons, and is believed to comprise a short polypeptide chain of about 50 kD which is responsible for the toxic properties of the toxin, and a larger polypeptide chain of about 100 kD which is believed to be necessary to enable the toxin to penetrate the nerve. The “short” and “long” chains are linked together by disulphide bridges.

With regard to these aforementioned, heretofore known uses, intramuscular injections of botulinum toxin A are generally used to balance muscle forces across joints, to diminish or decrease painful spasticity, to decrease deforming forces through selective motor paralysis, to diminish neuropathic and nociceptive pain, to diminish dystonic contractures, to decrease muscle deformation after injury or surgery, or to diminish sweating.

However, the use of appropriate doses of Botulinum neurotoxins (BoNT) to correct abnormal blood flow in disease states by the selective alteration of the components of microvascular flow using Botulinum A Toxin, other SNARE protein mediating agents, alpha adrenergic agents, and other compounds is heretofore unknown, whether delivered by injection or transdermal means. The invention discloses novel methods for the control of blood flow in skin, subcutaneous tissue and muscle by botulinum toxin, snare protein mediating substances, and alpha adrenergic agents by either injection or transdermal delivery systems.

BoNT type-A is currently administered intra-subdermally and injected in a grid pattern. Microvascular effects are secondary to diffusion and are not consistent or reliable. Direct delivery to interstitial channels at appropriate depths within the subdermal plexus and subdermal microvascular beds is shown to improve blood flow and is affected by the delivery systems and novel dosing as delineated herein. Alteration of the components of microvascular flow thereby improves inappropriate blood flow, decreases vasospasm, and maximizes regional blood flow, consequently diminishing symptoms and improving quality of life. It promotes healing after injury or surgery. In low temperature environments, this blood flow improvement will decrease painful effects and decreased dexterity secondary to reactive vasospasm, promote The Hunting Phenomenon, and improve performance during avocational and vocational activity.

Patients with collagen vascular disease and/or pain on exposure to cold and/or ulcers or sores experience increased vascular flow resulting in increased nutritional flow with a concomitant decrease in pain, Raynaud's phenomenon and cold sensitivity, and healing of refractory ulcers.

### SUMMARY OF THE INVENTION

Botulinum neurotoxin-A (BoNT-A) is a highly potent biological neurotoxin which blocks the pre-synaptic release of neurotransmitter from peripheral nerves. The motor effects of BoNT-A are well recognized but the vascular effects of this toxin are not known. Botulinum toxin is used to reduce sympathetic neural tone and obtain selective alteration and manipulation of the components of microvascular flow to improve function, decrease pain, and promote healing subsequent to disease, trauma, cold exposure, or surgery. Novel dosing regimens for delivery of botulinum toxins, snare protein mediating substance and alpha adrenergic agents and related compounds to acral skin beds and blood vessels are disclosed to effect manipulation of components of microvascular flow. Transdermal delivery systems facilitate delivery in non-hospital and clinical office environments, and by lay-persons, including self-administration.

Normal vascular control in striated muscle is mediated by the adrenergic nervous system, circulating vasoactive factors, and by local factors. Muscle tissue perfusion is based on the local metabolic demands of the tissue and the presence of hormones and neurotransmitters. Modulation of sympathetic nervous system tone is responsible in large part for the dilatory and contractile forces that comprise vascular tone. Release of the sympathetic neurotransmitter norepinephrine causes vasoconstriction by binding to alpha-adrenergic receptors on the vascular smooth muscle. The degree of vasoconstriction is positively associated with increasing levels of neurotransmitter in the synaptic space. Hence, vascular flow rates can be mediated by varying the frequency of the impulses through sympathetic nerves to effect changes in microvascular diameter.

Control of striated muscle microcirculation is effected both by systemic and local factors. Systemic control is mediated by humoral factors, both vasoconstrictive and vasodilatory, from many sources. The body also controls the striated muscle microvasculature through autonomic, sympathetic nerves located in the adventitia of blood vessels. These nerves are responsible for maintaining that portion of peripheral vascular resistance that is contributed by the vascular smooth muscle through the tonic release of norepinephrine, the vasoconstrictive neurotransmitter.

Botulinum neurotoxin-type A (BoNT-A) blocks synaptic neurotransmission by cleaving the pre-synaptic snare proteins required for neuronal vesicle fusion with the pre-synaptic neuron membrane and blocking the subsequent release of neurotransmitter. The target organelles contain soluble NSF attachment receptor (SNARE) proteins and neurotransmitter-containing vesicles which require these SNARE proteins for fusion of the vesicle to the cell membrane and release of neurotransmitter. Targets include neuromuscular junctions, sweat glands, vascular beds and nociceptors. This neural blockade usually is associated with skeletal muscle paralysis, but the toxin also blocks other forms of vesicular neurotransmission, such as the neurally-mediated release of acetylcholine required for sweat production. Sympathetically-mediated control of vascular resistance also can be impaired by BoNT-A wherein the action-potential-mediated release of norepinephrine from the autonomic sympathetic nerves present in the adventitia of arterioles, and to a smaller extent the veins, is blocked. Increases in blood flow are observed following vasodilation with BoNT-A. The topical application of BoNT-A to the muscle surface results in vasodilation due to blockade of sympathetic neural transmission.

It is an object of this invention to provide botulinum toxin and similar compounds and delivery systems to protect skin and exposed tissue from frostbite and/or exposure to extreme environments;

It is an object of the invention to utilize periadventitial application of BoNT to block vesicular release of vasoconstrictor elements from pre-synaptic neurons.

It is yet another object of this invention to provide compounds and delivery systems to improve and/or optimize vascular flow during wound healing after trauma or surgery;

It is an object of this invention to selectively manipulate components of microvascular flow to counter the effects of disease and improve tolerance on exposure to cold;

It is still another object of this invention to decrease pain on exposure to cold by selective control of microvascular to thereby maximize nutritional flow channels;

It is yet another object of this invention to provide injectable or transdermally applied means for increasing vascular flow using botulinum toxin and similar compounds; and,

It is an object of this invention to provide dosing regimens and delivery systems for botulinum toxin and similar compounds to improve the components of blood flow.

The invention will be better understood and objects other than those set forth above will become apparent when consideration is given to the following detailed description thereof. Such description makes reference to the annexed drawings wherein.

### BRIEF DESCRIPTION OF THE DRAWINGS

FIG. 1 is a graphical representation of experimental data showing change in the diameter of a rat cremaster arteriole when treated with botulinum toxin.

### DETAILED DESCRIPTION OF THE INVENTION

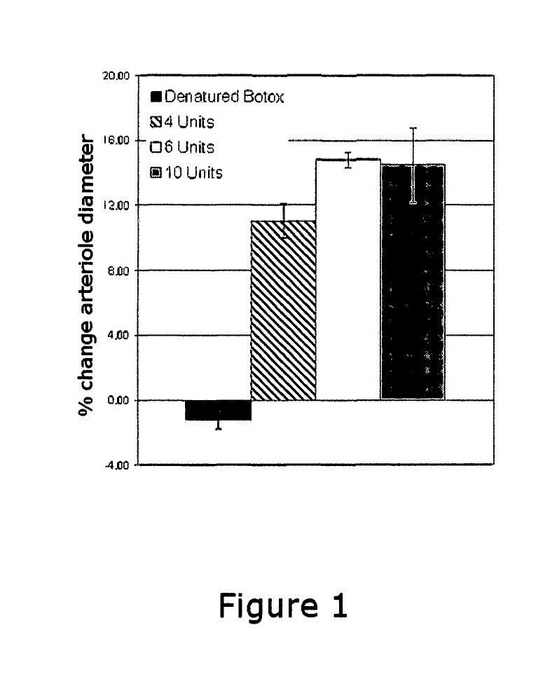

The preferred embodiment and best mode of the invention is shown in FIG. 1 and described in connection with certain preferred embodiments. However, it is not intended that the present invention be so limited. On the contrary, it is intended to cover all alternatives, modifications, and equivalent arrangements as may be included within the spirit and scope of the invention as defined by the appended claims.

In testing the efficacy of the present invention, male Sprague-Dawley rats (90-105 g) were selected for mammalian modeling and were anesthetized with an intramuscular injection of urethane at 120 mg/100 g. As is common practice in this field, the cremaster muscle was used because of its readily accessible microvasculature. Metabolic demands of the striated cremaster muscle were reduced prior to the application of BoNT-A by paralyzing the muscle with gallamine triethiodide to eliminate the possibility of confounding vascular changes due to decreased metabolic demands of the muscle tissue following BoNT-A treatment. This treatment allowed for a more accurate assessment of the BoNT-A effects on blood vessel diameter rather than the vascular responses to altered metabolic rate due to toxin-mediated muscle paralysis.

Subjects were placed on an acrylic stage containing a reservoir. The cremaster was exposed according to the Baez cremaster preparation technique (1972). The cremaster muscle was stretched over an adjustable, raised stage surrounded by a two piece reservoir. A notch in the upper portion of the reservoir allowed the pedicle of the muscle to remain free from pressure. The notch and seam in the reservoir were sealed with petroleum jelly (19-086291, Fisher Scientific, USA). The subject and platform were secured to the microscope stage in order to study the microvessels.

Small (about 50 μm-about 90 μm) arterioles were identified and pre-treatment microvascular diameters were recorded. Video measurements of the microvessels in the cremaster preparation were obtained using a Panasonic® CCD black and white camera (# KP-M1U) attached through a ⅔× Optim® extender to a Leitz® LaborLux 12 microscope with Zeiss® 40×LWD objective. Images were recorded on Panasonic® Professional S-VHS tapes with a Mitsubishi® S-VHS videocassette recorder. Vessels were measured from wall to wall using a video dimension micrometer. The micrometer was calibrated prior to the start of each experiment.

Once secured to the microscope, the bath and pump reservoir were filled with buffered modified Krebs solution (118.3 mM, 4.7 mM KCl, 2.5 mM CaCl₂, 1.2 mM MgSO₄, 1.2 mM KH₂PO₄, 25 mM NaHCO₃, 26.0 μM EDTA, 11.0 mM Glucose) for a total volume of 66 mL. A peristaltic pump (peristaltic pump and pressure servo control, Living Systems Instrumentation, Burlington, Vt. USA) provided circulation for the tissue bath at a rate of 49 μL/min. All drug solutions were added directly to the bath, room temperature was 23.3° C.

Arteriolar measurements were taken before and after the addition of the Krebs solution. Baseline measurements were taken again 10 minutes after the addition of 1 mg (0.170 mM) of the non-polarizing muscle relaxant gallamine triethiodide (Sigma-Aldrich, St. Louis, Mo.). Bottles of BoNT-A (100 Units; Allergan, Irvine, Calif.) were re-suspended in 1.0 mL 0.9% saline immediately prior to addition to the bath. The toxin was added directly to the bath near the cremaster muscle. As shown in FIG. 1, experimental subjects received one of three doses of BoNT-A: 4, 6 or 10 Units. Each Unit has a volume of 10 μL of saline solution. Vessels were measured immediately after the addition of the toxin and at 5 minute intervals for a 20 minute observation period. Any subsequent drug additions were made to the bath near the cremaster and were measured at 5 minute intervals.

Control subjects were treated with gallamine triethiodide, and 100 μL of saline solution (equivalent to the volume associated with 10 U BoNT-A) was added to the bath. After a 20 minute observation period, 10 U of denatured BoNT-A was added to the bath. Denaturation of BoNT-A was accomplished by placing the vial in boiling water for approximately 60 seconds until the BoNT-A solution changed from clear to cloudy.

All changes in vessels are the mean of the average change in observed vessels in each animal according to the treatments used. No significant effects on microvessel diameter were observed following the addition of Krebs solution to the tissue bath; however, all subjects exhibited vasoconstriction after the addition of gallamine triethiodide. Significant vasoconstriction was observed within 10 minutes after its addition to the tissue bath in all subjects.

The addition of BoNT-A to paralyzed muscle tissue results in vasodilation, with an observed increase in vessel diameter of 12-14% within 10 minutes after application according to the amounts of BoNT-A administered. A dose dependent response to BoNT application is shown, with saturation of the binding sites at a dose of 6 U BoNT-A. No significant change in the dilatory response is seen at concentrations greater than about 6 U BoNT-A. Control subjects exhibited no response to the addition of either saline solution (used for resuspension of BoNT-A) or denatured BoNT-A.

To ensure that there were no systematic time-dependent effects responsible for the observed vasodilation, control subjects were observed for a total of 50 minutes after the addition of gallamine triethiodide to the bath. No vasodilation was seen. Similarly, subjects treated with denatured BoNT-A following gallamine triethiodide were studied for 50 minutes after addition of the BoNT-A. No vasodilation was observed in this group during this time period.

Microvascular tone, the state of dilation/constriction reflecting the summation of all dilatory and constrictive influences in striated muscle also is controlled by local factors regulating perfusion of the muscle through a process termed autoregulation. Autoregulation controls blood flow into the striated muscle so that the perfusion of the muscle is matched to the metabolic demand for oxygen by the muscle, thus maximizing the efficiency of skeletal muscle perfusion. Gallamine triethiodide was administered to the striated muscle of the cremaster so that the metabolic effects of muscle paralysis (reduced oxygen consumption and vasoconstriction) were present prior to the administration of BoNT-A. Because the application of BoNT-A causes striated muscle paralysis in addition to blockade of autonomic sympathetic neurotransmission, the potential for confounding effects on the striated muscle microvasculature (i.e. autoregulatory vasoconstriction due to decreased metabolic and vasodilation due to blockade of the sympathetic neurons in the vascular adventitia) are avoided. As noted in the experimental results, the administration of gallamine triethiodide, a curareform paralytic agent that competitively antagonizes the post-synaptic acetylcholine receptors of the striated muscle, resulted in vasoconstriction, probably due to decreased metabolic demand of the paralyzed cremaster muscle. Cakmak et al., “Effect of Botulinum-A Toxin to Cremaster Muscle: An Experimental Study”, Urological Research, 2003 October; 31(5):352-4.

In vivo visualization and quantitation of microcirculatory diameter changes in the cremaster was conducted using intravital microscopy. Because this striated muscle is thin, intravital microscopy can be performed using transmitted light illumination. A recirculating tissue bath was used to approximate normal physiologic conditions and also provided a method for topical application of drugs in reasonable concentrations without damage to the muscle. Videomicroscopy permitted real-time observation of the vessel changes and all reactions to drug treatments were monitored in this way.

BoNT-A prevents the release of norepinephrine from nerve terminals responsible for innervating vascular smooth muscle thereby resulting in vasodilation whereby relaxation of the arterial smooth muscle increases the vessel diameter. Once the sites on the sympathetic endings are saturated or all available BoNT-A reacted, dilation ceases. Maximal vasodilation was generally seen within 10 minutes of BoNT-A application and was sustained for the duration of the trial.

The observed vasodilation from treatment with BoNT-A resulted in a significant change in blood flow to the surrounding tissue. Since the topical application of BoNT to the cremaster does not elicit an alteration in systemic arterial pressure, the blood flow through the cremasteric arterioles was necessarily increased. Increases of 12-14% in vessel diameter cause concurrent increases of 25-30% in cross-sectional area. However, since Poiseuille's law determines resistance to blood flow, the true reduction in resistance to flow is a function of the radius of the blood vessel to the fourth power, assuming that the arterial pressure is unchanged. Therefore, with an unchanged systemic arterial pressure, a 14% increase in radius results in a 69% reduction in resistance with a concomitant 69% increase in blood flow. In addition, the cremaster muscle was paralyzed with gallamine triethiodide (as well as with Botulinum neurotoxin) thereby reducing the metabolic demand of the muscle tissue. The observed increase in perfusion is therefore paradoxical in light of normal autoregulation (both metabolic and myogenic) of the blood flow to the cremaster, which would be expected to reduce perfusion to match the metabolic requirements of the now-paralyzed muscle.

The use of BoNT-A to increase blood flow has important clinical implications in the treatment of vasculature diseases, such as Raynaud's phenomenon. Patients suffering from Raynaud's phenomenon experience a loss of blood flow in digits, especially in cold temperatures. Increasing the vessel size, and thus blood flow, allows patients to resume activity. BoNT-A may also be useful in treating Mixed Connective Tissue Disease and scleredoma.

Returning to FIG. 1, following treatment with BoNT-A, vasodilation was observed in small arterioles. The degree of dilation varied with the number of BoNT-A units applied. As a result of the vasodilation, flow rate increased in the vessels. This observation has important clinical implications in the treatment of peripheral vascular diseases. In their preferred embodiment, the compounds are delivered by injection or via transdermal means, including but not limited to transdermal patches, ointments, creams, lotions, gels, sprays, or other topical application methods.

Normal vascular control is mediated, in part, by the adrenergic system. Muscle tissue perfusion is based on the local metabolic demands of the tissue and the presence of hormones and neurotransmitters. Sympathetic innervation is responsible for the dilatory and contractile forces that comprise vascular tone. Release of the sympathetic neurotransmitter norepinephrine causes vasoconstriction by binding to alpha-adrenergic receptors. The amount of vasoconstriction parallels increases in the amount of neurotransmitter released. Topical application of BoNT-A provides prolonged vasodilation of specific vascular beds following targeted application.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What I claim is:

1. A method for increasing vascular flow in a mammal comprising the steps of:
selecting a target region of the human body;
determining the dosage of botulinum toxin necessary to effect vasodilation in the target region; and,
delivering said dosage of botulinum toxin adjacent the vasculature of said target region to increase blood flow through the blood vessels.

2. The method for increasing vascular flow as claimed in claim 1 wherein said dose of botulinum toxin is a titrated dose wherein the smallest effective dose is selected to achieve the desired vasoconstriction.

3. The method for increasing vascular flow as claimed in claim 1 wherein said dose of botulinum toxin is delivered topically.

4. The method for increasing vascular flow as claimed in claim 3 wherein said topically delivered botulinum toxin is selected from the group of botulinum-containing compounds consisting of pastes, ointments, creams, lotions, gels, and sprays.

5. The method for increasing vascular flow as claimed in claim 1 wherein said dose of botulinum toxin is delivered transdermally.

6. The method for increasing vascular flow as claimed in claim 5 wherein said transdermally delivered botulinum toxin is selected from the group of botulinum-containing compounds consisting of pastes, ointments, creams, lotions, gels, and sprays.

7. The method for increasing vascular flow as claimed in claim 5 wherein said dose of botulinum toxin is contained in a transdermal patch.

8. The method for increasing vascular flow as claimed in claim 1 wherein said dose of botulinum toxin is injected.

9. The method for increasing vascular flow as claimed in claim 1 wherein said botulinum toxin is botulinum toxin type A

10. The method for increasing vascular flow as claimed in claim 1 wherein said botulinum toxin is botulinum toxin taken from a group consisting of type B, C, D, E, and F.

11. A method for controlling blood flow in skin, subcutaneous tissue, and muscle comprising the steps of:
selecting a target region of a mammal body;
determining the dosage medically effective of botulinum toxin necessary to effect vasodilation in the target region; and,
delivering a dosage of botulinum toxin transdermally to the vasculature of said target region to increase the cross sectional area of blood vessels 25% to 30% in cross sectional area.

12. The method for controlling blood flow in skin, subcutaneous tissue, and muscle as claimed in claim 11 wherein said dose of botulinum toxin is a titrated dose wherein the smallest effective dose is selected to achieve the desired vasoconstriction.

13. The method for controlling blood flow in skin, subcutaneous tissue, and muscle as claimed in claim 11 wherein said transdermally delivered botulinum toxin is selected from the group of botulinum-containing compounds consisting of pastes, ointments, creams, lotions, gels, and sprays.

14. The method for controlling blood flow in skin, subcutaneous tissue, and muscle as claimed in claim 11 wherein said dose of botulinum toxin is contained in a transdermal patch.

15. The method for increasing vascular flow as claimed in claim 11 wherein said botulinum toxin is botulinum toxin type A

16. The method for increasing vascular flow as claimed in claim 11 wherein said botulinum toxin is botulinum toxin taken from a group consisting of type B, C, D, E, and F.

17. A method for controlling blood flow in skin, subcutaneous tissue, and muscle comprising the steps of:
selecting a target region of a mammal body;
determining the dosage medically effective of botulinum toxin necessary to effect vasodilation in the target region; and,
delivering a dosage of botulinum toxin directly to the vasculature of said target region to increase the cross sectional area of blood vessels 25% to 30% in cross sectional area.

18. A method for controlling blood flow in skin, subcutaneous tissue, and muscle comprising the steps of:
selecting a target region of a human body;
selecting a botulinum toxin type A containing transdermal patch, paste, ointment, creams, lotion, gel, or spray;
determining the dosage of botulinum toxin medically effective and necessary to effect vasodilation in the target region; and,
delivering at least one dose of botulinum toxin via said transdermal patch, paste, ointment, creams, lotion, gel, or spray transdermally to the vasculature of said target region to increase blood vessel diameter size between 10% to 15% and blood flow within treated blood vessels over 50%.

19. The method for controlling blood flow in skin, subcutaneous tissue, and muscle as claimed in claim 18 wherein said dose of botulinum toxin is a titrated dose wherein the smallest effective dose is selected to achieve the desired vasoconstriction.

20. The method for controlling blood flow as claimed in claim 18 wherein said target region has frostbite.

21. The method for controlling blood flow as claimed in claim 18 wherein said dosage ranges from about 4 Units of BoNT-A to about 6 Units of BoNT-A

22. The method for controlling blood flow as claimed in claim 18 wherein said vasodilation ranges from about 12% to about 14% after about 10 minutes.

23. The method for controlling blood flow as claimed in claim 18 wherein said blood vessels increase from 25%-30% in cross sectional area.
